(19) United States
(12) Patent Application Publication
(10) Pub. No.: US 2023/0148946 A1
(43) Pub. Date: May 18, 2023

(54) METHODS OF DETECTING ALLERGIC REACTIONS IN SUBJECTS USING MICROCHANNEL DELIVERY DEVICES

(71) Applicant: AQUAVIT PHARMACEUTICALS, INC., Las Vegas, NV (US)

(72) Inventor: Sobin Chang, Las Vegas, NV (US)

(21) Appl. No.: 17/915,090

(22) PCT Filed: Mar. 26, 2021
(86) PCT No.: PCT/US2021/024478

§ 371 Date: Sep. 27, 2022

Related U.S. Application Data
(60) Provisional application No. 63/001,153, filed on Mar. 27, 2020.

(51) Int. Cl. A61B 5/00; A61M 37/00

(52) U.S. Cl. CPC A61B 5/411; A61M 37/0015; A61M 2037/003; A61M 2037/0061

(57) ABSTRACT

The present invention provides methods for determining whether a subject is allergic to an allergen, comprising administering to the subject's skin at an injection site a composition comprising the allergen, wherein the composition is administered with a microneedle delivery device; and monitoring the injection site of the subject's skin for the appearance of a hypersensitivity reaction, wherein the appearance of the hypersensitivity reaction indicates that the subject is allergic to the allergen.

### CROSS-REFERENCE TO RELATED APPLICATIONS

This application claims the benefit of U.S. Provisional Application No. 63/001,153, filed Mar. 27, 2020, the contents of which are incorporated by reference herein in its entirety.

### FIELD OF THE INVENTION

The field of the invention relates generally to the field of medicine, medical devices, and immunology and allergic disease, specifically methods and devices useful for detecting allergic reactions in subjects.

### BACKGROUND OF THE INVENTION

Normally, allergy symptoms are caused by the interaction between an allergen and an antibody known as IgE (immunoglobulin E). To diagnose an allergy, an allergist generally uses a skin prick test (SPT) to measure the presence of IgE antibodies for the suspected allergy.

During this test, the allergist generally places a drop of solution containing the allergen on the subject's forearm or back. Using a small needle, the doctor gently pricks or scratches the skin to allow a tiny amount of the solution to enter just below the surface. The SPT is generally not painful and there is no bleeding. The scratching on the surface of the skin feels similar to a fingernail scratch.

Positive results are indicated by a wheal—a raised white bump surrounded by a small circle of itchy red skin. In general, a large wheal is more likely to indicate a true allergy, but the size is not always an accurate predictor.

### SUMMARY OF THE INVENTION

It is to be understood that both the foregoing general description of the embodiments and the following detailed description are exemplary, and thus do not restrict the scope of the embodiments.

In one aspect, the invention provides a method for determining whether a subject is allergic to an allergen, comprising
i) administering to the subject's skin at an injection site a composition comprising the allergen, wherein the composition is administered with a microneedle delivery device; and
ii) monitoring the injection site of the subject's skin for the appearance of a hypersensitivity reaction,
   wherein the appearance of the hypersensitivity reaction indicates that the subject is allergic to the allergen.

In some embodiments, the allergen is from a substance shown in Table 1. In some embodiments, the allergen is a polypeptide.

In some embodiments, the injection site is monitored for at least 15 minutes.

In some embodiments, the injection site is monitored for at least 30 minutes.

In some embodiments, the injection site is monitored for at least one hour.

In some embodiments, the administering comprises a repeated motion of penetrating the microneedle delivery device into the subject's skin at the injection site.

In some embodiments, a plurality of compositions are administered to the subject's skin at a plurality of injection sites.

In some embodiments, a plurality of microneedle delivery devices are used to administer the compositions.

In some embodiments, the plurality of injection sites are arranged in a grid format on the subject's skin.

In some embodiments, the injection sites are located on the subject's back and/or forearm.

In some embodiments, the microneedle delivery device comprises
i) one or more microneedles, wherein the microneedles are hollow or non-hollow, wherein one or multiple grooves are inset along an outer wall of the microneedles; and
ii) a reservoir that holds the composition to be delivered, wherein the reservoir is attached to or contains a means to encourage flow of the composition contained in the reservoir into the skin;

wherein the composition is delivered into the skin by passing through the one or multiple grooves along the outer wall of the microneedle.

the microneedles are non-hollow.

In some embodiments, the means to encourage flow of the composition contained in the reservoir into the skin is selected from the group consisting of a plunger, pump and suction mechanism.

In some embodiments, the means to encourage flow of the composition contained in the reservoir into the skin is a mechanical spring loaded pump system.

In some embodiments, the microneedles have a single groove inset along the outer wall of the microneedle, wherein the single groove has a screw thread shape going clockwise or counterclockwise around the microneedle.

In some embodiments, the microneedles are from 0.1 mm to about 2.5 mm in length and from 0.01 mm to about 0.05 mm in diameter.

In some embodiments, the microneedles are made from a substance comprising gold.

In some embodiments, the plurality of microneedles comprises an array of microneedles in the shape of a circle.

In some embodiments, the microneedles are made of 24-carat gold plated stainless steel and comprise an array of 20 microneedles.

In some embodiments, the method further comprises administering to the subject a treatment for the allergy. In some embodiments, the treatment comprises administering to the subject an effective amount of an immunosuppressant.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1 is a view of a handheld microneedle injection apparatus. The syringe ejection volume is automatically controlled and dispenses into an interchangeable head containing one or several needles. The diagram shows the connection of corrugated connector and microneedle head. The rubber based connector is such that its flexibility will allow connections with small openings (1) and large ones (2) to fit and seal the microneedle head. The corrugated connector, also made of rubber (3), will further allow larger embodiments to connect to this system with the spring plate microneedle head (4).

FIG. 2 is an image of a screw on a microneedle head.

FIG. 3 is a schematic representation of a device in a syringe configuration. Alternative configurations include vial- and capsule-loaded configurations. The device holds a syringe (2) for automatic injection via one or more microneedles in the microneedle head. Ejection volume is controlled by an information processor (9). Other elements are noted: the motor or actuator (4) to control the piston (3), exchangeable and controllable needle head (1) and cam system and dial to adjust needle injection depth (5), and needle head ejector (10). Information is shown to the user in a display panel that may include a manual or touchscreen control panel (12) and data is stored in a storage unit (11) that may be removable. The needle head (1) may be controlled by an actuator (13).

FIG. 4 provides three additional views of a microneedle device. Microneedle components: (A) microneedles, (B) housing of the needles and (C) a reservoir.

FIG. 5 is a diagram showing the connection of corrugated connector and microneedle head. The rubber based connector is such that its flexibility will allow connections with small openings (1) and large ones (2) to fit and seal the microneedle head. The corrugated connector, also made of rubber (3), will further allow larger embodiments to connect to this system with the spring plate microneedle head (4).

FIG. 6 provides a depiction of the utility feature conferred by the circular or flat O-Rings. Said features enable enhanced liquid handling capabilities as evidenced by an airtight mechanism which facilitates the efficient and uniform delivery of treatment solutions to the skin. Said features are positioned at the interface of the cap and the reservoir channel so as to effectively prevent the leakage of treatment solution dosages. The RFID chip+O-ring depiction has been expanded. The cap/cover (1) will interface with the vial or container (5) containing a certain compound (6). The connection of both the cap/cover and the container may be sealed with a threaded opening (2). While pressure is applied vertically through the twisting motion of the thread, the rubber O-ring (3) seals the two interfaces (1) and (5) together. A ratchet mechanism (4) at the end will lock the cap in place. Embedded inside the rubber O-ring is a RFID chip (7) which material is shock, pH, temperature, and ozone resistant. The RFID chip will be stable enough under different environments to be able to effectively transmit data for applications such as data security, quality assurance/control, and logistics (8).

FIG. 7A-7B depict a utility feature conferred by the circular or flat O-Rings (FIG. 7A). Said features enable obvious and non-obvious advantages conferred by excellent weather and ozone resistance, temperature resistance (FIG. 7B) and the resistance to pH induced degradation of the butyl rubber or halogenated butyl rubber in comparison to other industrial rubbers and further addresses the stability of the material in the context of medical device utility, end user performance and pharmacological agent turbidity. Said features effectively enable enhanced material durability while preventing the leakage and inefficient delivery of treatment solution dosages with time.

FIG. 8 illustrates anti-unlock safety features of an O ring in a microneedle device.

FIG. 9 illustrates anti-unlock safety features of an O ring in a microneedle device.

FIG. 10 illustrates anti-unlock safety features of an O ring in a microneedle device.

FIG. 11 illustrates anti-unlock safety features of an O ring in a microneedle device.

FIG. 12 illustrates an exemplary microneedle drug delivery device.

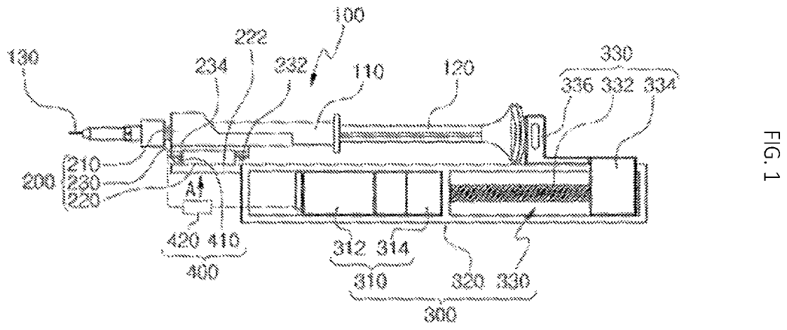

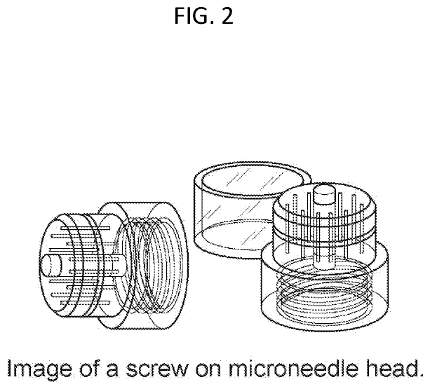

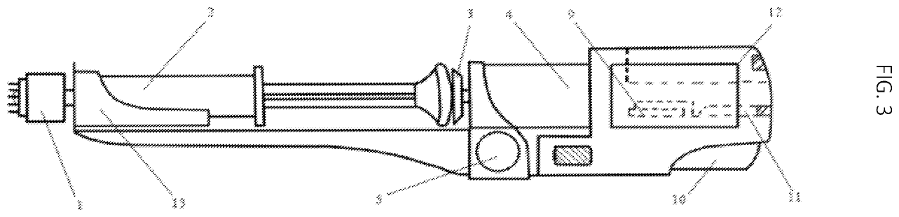

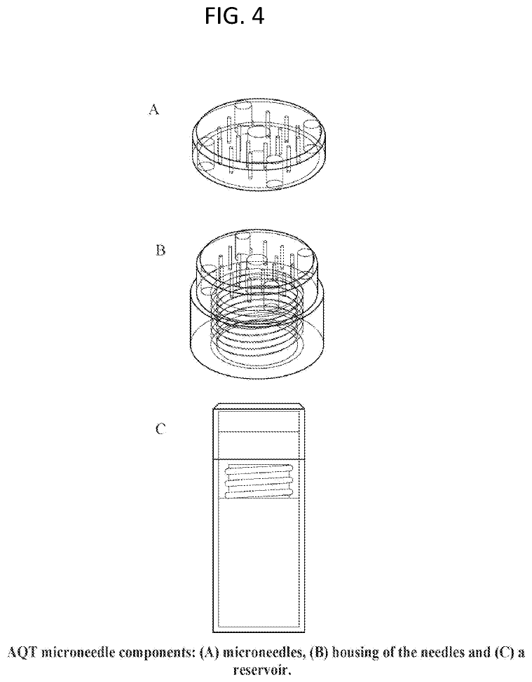

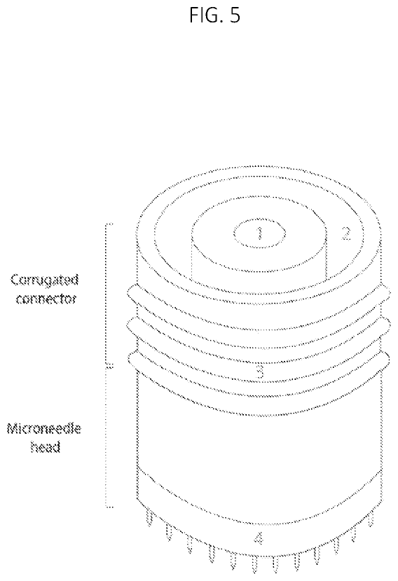

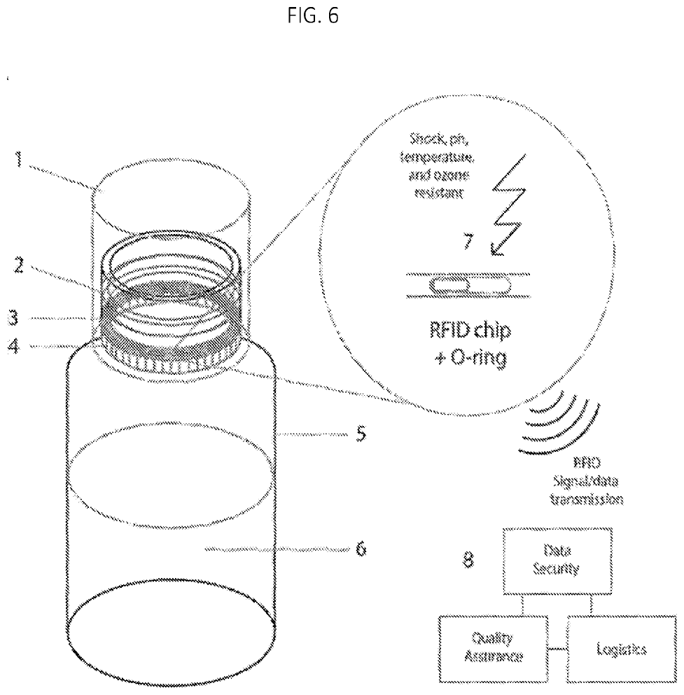

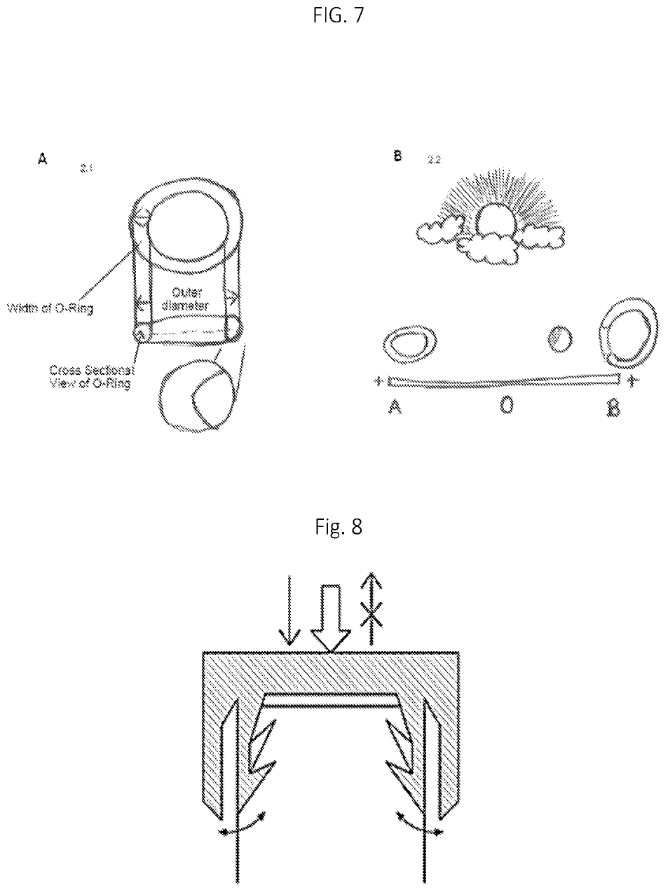

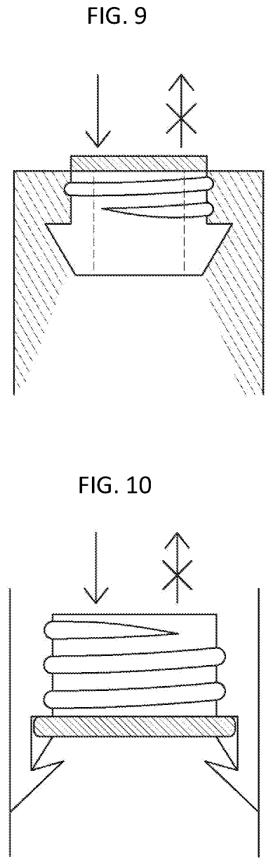

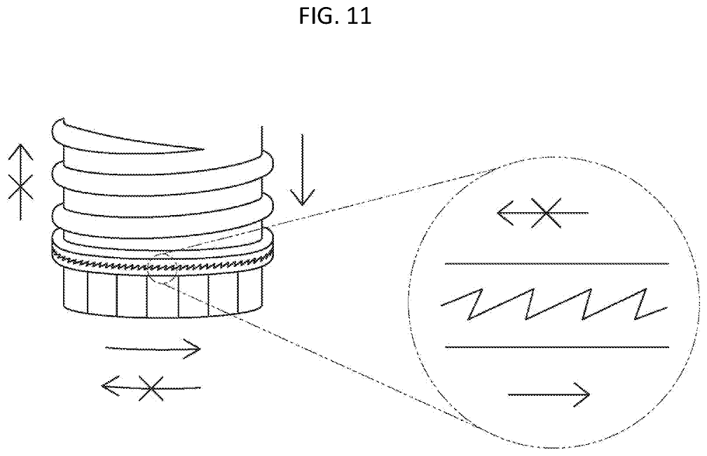

### DETAILED DESCRIPTION OF THE INVENTION

Reference will now be made in detail to the presently preferred embodiments of the invention which, together with the drawings and the following examples, serve to explain the principles of the invention. These embodiments are described in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.
In one embodiment, the invention provides a method for determining whether a subject is allergic to an allergen, comprising
i) administering to the subject's skin at an injection site a composition comprising the allergen, wherein the composition is administered with a microneedle delivery device; and
ii) monitoring the injection site of the subject's skin for the appearance of a hypersensitivity reaction,
wherein the appearance of the hypersensitivity reaction indicates that the subject is allergic to the allergen.

The term “subject” as used herein is not limiting and is used interchangeably with patient. In some embodiments, the term subject refers to animals, such as mammals and the like. For example, mammals contemplated include humans, primates, dogs, cats, sheep, cattle, goats, pigs, horses, chickens, mice, rats, rabbits, guinea pigs, and the like.

The composition comprises an allergen. In some embodiments, the allergen is a purified substance. In some embodiments, the allergen comprises a mixture of substance. In some embodiments, the allergen is a crude isolate or preparation. The allergen that can be used in the invention is not limiting. Sources of potential allergens are shown below in Table 1.

### Table 1. Allergen Sources.

SEASONAL INHALANTS

Grasses
Bahia Grass
Barley Grass
Bermuda Grass
Brome, Smooth
Canary Grass, Reed
Corn, Cultivated
Fescue, Meadow
Foxtail, Meadow
Johnson Grass
Kentucky Bluegrass
Oat, Cultivated
Orchard Grass
Red Top, Bentgrass
Reed, Common
Rye Grass
Rye Grass, Perennial
Rye Grass, Wild
Salt Grass
Sweet Vernal
Timothy
Velvet Grass
Wheat, Cultivated

Trees
Alder, Grey
Ash, White
Bayberry/Sweet Gale
Beech, American
Cedar, Mountain
Cedar, Red
Common Silver Birch
Cottonwood
Cypress, Bald
Cypress, Italian
Elm, American
Elm, Cedar
Eucalyptus
Fir, Douglas
Gum, Sweet
Hackberry
Hazelnut Tree
Hickory, White
Hop-Hornsbeam
Japanese Cedar
Lime Tree/Linden
Maple/Box Elder
Melaleuca
Mesquite
Mimosa/Acacia
Mulberry, White
Oak, Live/Virginia
Oak, White
Olive, Russian
Olive Tree
Palm, Queen
Pecan, Hickory
Pepper Tree
Pine, White
Privet, Common
Spruce
Sycamore, Maple Leaf
Walnut (Pollen)
Willow

Weeds
Alfalfa
Careless Weed
Cocklebur
Dandelion
Dockweed, Yellow
Fennel, Dog
Goldenrod
Kochia
Lamb's Quarters
Lenscale
Mugwort
Nettle
Pigweed, Common
Plantain, English
Ragweed, False
Ragweed, Giant
Ragweed, Short
Ragweed, Western
Rough Marshelder
Sheep Sorrel
Sunflower Pollen
Thistle, Russian
Wall Pellitory
Wormwood

NONSEASONAL INHALANTS

Animals
Animal Mix (Cat, Cow, Dog, Horse)
Budgerigar Droppings
Budgerigar Feather
Canary Feathers
Cat Hair/Dander
Chicken Feathers
Cow Dander
Dog Dander
Duck Feathers
Finch Feathers
Gerbil Epithelium
Goat Epithelium
Goose Feathers
Guinea Pig Epithelium
Hamster Epithelium
Horse Dander
Mouse Epithelium
Mouse Serum
Mouse Urine
Pigeon Feathers
Rabbit Epithelium
Rat Epithelium
Rat Epithelium and Protein
Rat Serum
Rat Urine
Sheep Epithelium
Swine Epithelium
Turkey Feathers

BACTERIA
Staphylococcus Enterotoxin A
Tilletia tritici

CHEMICALS/OCCUPATIONAL
Alpha-Amylase
Castor Bean
Cotton Lint
Ethylene Oxide
Ficus
Formaldehyde
Isocyanate HDI
Isocyanate MDI
Isocyanate TDI
Ispaghula
Latex
Oxeye Daisy
Papain
Phthalic Anhydride
Seminal Fluid
Silk
Sunflower Seed
Tobacco Dust
Trimellitic Anhydride

DRUGS
Adrenocorticotropic Hormone (ACTH)
Cefaclor
Chymopapain
Gelatin
Insulin, Bovine
Insulin, Human
Insulin, Porcine
Penicilloyl G
Penicilloyl V
Suxamethonium

HOUSE/DUST MITES
Acarus Mite
Blomia tropicalis
Dermatophagoides farinae
Dermatophagoides pteronyssinus
Euroglyphus maynei
Glycyphagus domesticus
Lepidoglyphus
Tyrophagus

INSECTS-WHOLE BODY
Cockroach, American
Cockroach, German
Fire Ant (Invicta)
Horse Fly
Midge
Moth
Whole Body: Mosquito

INSECTS-VENOM
Bumblebee
Honeybee
Hornet, White Face
Hornet, Yellow
Wasp, Paper
Yellow Jacket

MOLDS
Acremonium kiliense
Alternaria alternata
Aspergillus fumigatus
Aspergillus niger
Aureobasidi pullulans
Bipolaris
Botrytis cinerea
Candida albicans
Chaetomium globosum
Cladosporium herbarum
Curvularia lunata
Epicoccum purpur
Fusarium proliferatum
Mold Mix 1 (Alternaria alternata, Aspergillus fumigatus, Cladosporium herbarum, Penicillium chrysogenum)
Mucor racemosus
Penicillium chrysogenum
Phoma betae
Pityrosporum orbiculare
Rhizopus nigricans
Setomelanomma rostrata
Stachybotrys atra
Stemphylium herbarum
Trichoderma viridae
Trichophyton mentagro
Trichophyton rubrum

PARASITES
Ascaris

FOOD
Acacia Gum
Allspice
Almond
Alpha-Lactalbumin
Anchovy
Aniseed
Apple
Apricot
Asparagus
Avocado
Banana
Barley
Basil
Bayleaf (Laurel)
Beef
Beta-Lactoglobulin
Bing Cherry
Black Peppercorn
Blackberry
Blueberry
Boiled Milk
Brazil Nut
Broccoli
Brussels Sprout
Buckwheat
Cabbage
Carmine Red Dye
Carob Bean
Carrot
Casein
Cashew Nut
Catfish
Cauliflower
Celery
Cheese, Cheddar Type
Cheese, Mold Type
Chicken
Chick Pea
Chili Pepper
Chocolate/Cacao
Cinnamon
Clam
Cloves
Coconut
Codfish
Coffee
Coriander/Cilantro
Corn
Cottonseed
Crab
Cranberry
Crayfish, Freshwater
Cucumber
Cumin
Curry Powder
Date
Dill
Egg White
Egg, Whole
Egg Yolk
Eggplant
Fennel, Fresh
Fig
Flounder
Garlic
Ginger
Gluten
Goat's Milk
Grape
Grapefruit
Green Bean
Green Peppercorn
Green Pea
Haddock
Hake
Halibut
Hazelnut (Filbert)
Herring
Honey
Hop (Food)
Kidney Bean
Kiwi Fruit
Lamb
Lemon
Lentil
Lettuce
Lima Bean
Lime
Linseed
Lobster
Macadamia Nut
Mackerel
Malt
Mango
Melon
Milk
Millet
Mint
Mushroom
Mussel
Mustard
Nutmeg
Oat
Olive, Black
Onion
Orange
Oregano
Ovalbumin
Ovomucoid
Oyster
Papaya Food
Paprika/Sweet Pepper
Parsley
Peach
Peanut
Pear
Pecan Nut
Pine Nut, Pignoles
Pineapple
Pistachio Nut
Plum
Poppy Seed
Pork
Potato, White
Pumpkin
Rabbit Meat
Raspberry
Red Beet
Red Snapper
Rice
Rye
Sage, Salvia
Salmon
Sardine
Scallop
Sesame Seed
Shrimp
Sole
Soybean
Spinach
Squid
Strawberry
Sweet Chestnut
Sweet Potato
Swordfish
Tangerine
Tea
Thyme
Tilapia
Tomato
Trout
Tuna
Turkey
Vanilla
Walleye Pike
Walnut
Watermelon
Wheat
Whey
White Bean
Whitefish
Yeast

In some embodiments, the process comprises the following steps.

1. A grid sketch template is placed over the skin. This will create a grid pattern containing 20 to 50 squares

2. The physician aligns the microneedle device to the grid pattern and presses it over the skin

3. After administration, the device is disposed

4. After about 30 minutes, the physician conducts visual observation on each square to identify and determine the severity of individual allergens

5. If wheals are observed, the grid in the skin is correlated to the sheet containing the grid of allergens

6. If positive results are results are identified, the physician informs the patient

In some embodiments, the results are analyzed using a validated scale within minutes. In some embodiments, one or more treatments for the allergy are administered to the subject.

In some embodiments, the subject is administered one or more active agents to treat the allergy. In some embodiments, one or more of Antihistamines, Corticosteroids, Mast cell stabilizers (e.g., cromolyn sodium), Leukotriene inhibitors, Nasal anticholinergics, Decongestants, Immunomodulators, or Autoinjectable epinephrine are administered.

In some embodiments, the agent is selected from one or more of the following agents:
diphenhydramine (Benadryl)
chlorpheniramine (Chlor-Trimeton)
Brompheniramine (Dimetapp, Dimetane)
Carbinoxamine (Palgic)
Clemastine (Tavist)
Cyproheptadine (Periactin)
Hydroxyzine (Vistaril)
Cetirizine (Zyrtec, Zyrtec-D)
Clemastine (Tavist)
Fexofenadine (Allegra, Allegra D)
Loratadine (Claritin, Claritin D, Alavert)
Antazoline/Naphazoline (Vasocon A)
Ketotifen fumarate (Zaditor, Alaway, Zyrtec, Claritin Eye and Refresh Eye)
Naphazoline/Pheniramine (Naphcon-A, Opcon-A, Visine A)
Cromolyn (Opticrom)
Oxymetazoline (Afrin)
Triamcinolone acetonide (Nasacort)
Fluticasone propionate (Flonase)
Budesonide (Rhinocort)
Cromolyn sodium (Nasalcrom)
Acrivastine (Semprex-D)
Carbinoxamine (Palgic)
Cyproheptadine (Periactin)
Desloratadine (Clarinex)
Hydroxyzine (Vistaril)
Levoceterizine (Xyzal)
Azelastine (Optivar)
Emadastine (Emadine)
Epinastine (Elestat)
Olopatadine (Patanol, Pataday, Pazeo)
Cromolyn Sodium (Intal)
Lodoxamine (Alomide)
Nedocromil (Alocril)
Pemirolast (Alamast)
Loteprednol (Alrex)
Ketorolac (Acular)
Azelastine (Astelin, Astepro)
Azelastine and fluticasone (Dymista)
Olopatadine (Patanase)
Beclomethasone dipropionate (QNasl)
Budesonide (Rhinocort)
Ciclesonide (Omnaris, Zetonna)
Flunisolide
Fluticasone furoate (Veramyst)
Fluticasone propionate (Flonase)
Ipratropium Bromide (Atrovent)
Prednisone
Prednisolone
Cortisol
Methylprednisolone
Montelukast (Singulair)
Tacrolimus (Protopic Ointment)
Pimecrolimus (Elidel Cream)
In some embodiments, the subject is administered an effective amount of an immunosuppressant. In some embodiments, the specific treatment is personalized to the subject depending on their profile of allergies. In some embodiments, one or more treatments are administered using a microneedle delivery device. In some embodiments, the treatment formulation or composition is compounded using an automated device based on the subject's allergic profile. In some embodiments, a treatment based on the subject's allergic profile is determined using one or more algorithms. In some embodiments, the subject's assessment is made on site for example by a heath care provider or allergist. In some embodiments, an artificial intelligence assisted compounding device can be used to formulate a treatment. In some embodiments, the artificial intelligence assisted compounding device can monitor the treatment and optimize it over time for optimal personalization. See, e.g., U.S. Pat. No. 9,675,519 which is incorporated by reference in its entirety.

A system and method for personalized injection treatment can be synchronized with a complementary health management system (a computerized and networkable via wireless or wired connection) and can automatically compound and record patient specific doses of injectable medicine for disease prevention and/or treatment and the patient's progress will be monitored on the Internet longitudinally. The term herein can mean a sick or healthy human or animal (e.g., mammals such as dogs, cats, pigs, horses or cattle). The term “pharmaceutical agent” as used herein refers to an injectable solution providing health benefits and/or a therapeutic effect and or therapeutic potency including, but not limited to a vitamin, a drug, a neutraceutical, a biologic or any combination thereof. In some embodiments, the system can compound the components with the customized, personalized dosage proportion, and then prepare a single product container such as a product vial or syringe for injection. The size can be similar to those gourmet single cup coffee machines or inkjet/laser printers. The proportion is determined by the physician or an Internet tool that is scientifically validated. A physician may make a personalized compounding order for injectable solutions for treatment of allergic conditions, and through communication devices such as a computer, a PDA, or a smart phone. Operators such as physicians or nurses may input a compounding order directly and manually with using a control pad and the system may have a control screen such LED and LCD monitor. Alternately, the system can have a touch screen unit combining display and input units. When the single product container is created, the optimizer will input the patient record into the server simultaneously. The patient may have received a set of codes in advance via an Internet tool and may bring in the print-out for the physician. An inventory structure can have a plurality of vertical chambers individually holding one or more pharmaceutical agent-containing capsules.

Each chamber can typically store 10 to 30 capsules. The capsules can have a volume capacity of about 0.1 to about 10.0 mL, preferably 0.1 mL to about 2.0 mL liquid, and most preferably about 1.0 mL liquid. The pharmaceutical agent-containing capsules and the chambers preferably will be clearly labeled and color coded. Once the pharmaceutical agent-containing capsules are loaded into the device, it will compound the exact proportion into a single injection product container. The delivery components are thoroughly sterilized and an empty product container can be loaded by an operator. The product container can have a volume capacity of about 0.1 to about 10.0 mL and typically about 2.0 mL liquid. The capsules and product container can be completely or partially filled with the pharmaceutical agent. Depending on the compounding order, the capsules can be selectively loaded and transported to a processing area. At the processing area, the selected capsules can be connected to the product container and a quantity of the predetermined pharmaceutical agent can be transferred to the product container under positive or negative pressure. The positive pressure can be generated by mechanical compress outside the selected capsules and the negative pressure inside the selected capsules can be employed by a vacuum pump or syringe in order to draw the pharmaceutical agent out of the capsule. Alternatively, the capsules can be properly pressurized and packaged so that inside solution may automatically come out without auxiliary pressurization. The transferring quantity can be controlled by regulating amount of the applied pressure and time. The consumed medicine capsules are disposed of automatically after a single use. The solution inside the capsules can be transferred through a connector, which is pre-assembled with a product vial as a cap before it is placed into the system and consisted of a needle and an air vent. Alternatively, the solution can be transferred through an extraction needle engaged with the loaded capsules and an injection system engaged with the product vial. The system may be enclosed by a housing and an air ventilation system may be included in the apparatus. All of the embodiments described below can include sterilization means such as UV lamps to provide an internal aseptic environment. A plurality sensors such as proximity sensors, distance sensors, and temperature sensors may be placed in order to monitor if desired capsules are loaded, in order to monitor if the system operates properly in order to check if the gate/door is closed, and also in order to monitor temperature inside the system. Sensors can be used in each of the embodiments described below and are commercially available, for example, from Sharp in Japan, Keyence Corporation of America in Itasca, Ill., Automation Direct in the U.S., and c3 controls in the U.S. After all processes are completed, the product container can be taken out by an operator. Labels which include patient information and compounding order with text, bar code, or QR code format, can be printed out and the labels will be attached on the product container and the patient chart.

In some embodiments, the allergen comprises a polypeptide or an antigenic fragment thereof. An antigenic fragment is a polypeptide having an amino acid sequence that entirely is the same as part but not all of the amino acid sequence of one of the polypeptides. The antigenic fragment can be “free-standing,” or comprised within a larger polypeptide of which they form a part or region, most preferably as a single continuous region.

In some embodiments, the antigenic fragments include, for example, truncation polypeptides having the amino acid sequence of the polypeptides, except for deletion of a continuous series of residues that includes the amino terminus, or a continuous series of residues that includes the carboxyl terminus or deletion of two continuous series of residues, one including the amino terminus and one including the carboxyl terminus. In some embodiments, fragments are characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, and high antigenic index regions.

The fragment can be of any size. An antigenic fragment is capable of inducing an immune response in a subject or be recognized by a specific antibody. In some embodiments, the fragment corresponds to an amino-terminal truncation mutant. In some embodiments, the number of amino terminal amino acids missing from the fragment ranges from 1-100 amino acids. In some embodiments, it ranges from 1-75 amino acids, 1-50 amino acids, 1-40 amino acids, 1-30 amino acids, 1-25 amino acids, 1-20 amino acids, 1-15 amino acids, 1-10 amino acids and 1-5 amino acids.

In some embodiments, the fragment corresponds to carboxyl-terminal truncation mutant. In some embodiments, the number of carboxyl terminal amino acids missing from the fragment ranges from 1-100 amino acids. In some embodiments, it ranges from 1-75 amino acids, 1-50 amino acids, 1-40 amino acids, 1-30 amino acids, 1-25 amino acids, 1-20 amino acids, 1-15 amino acids, 1-10 amino acids and 1-5 amino acids.

In some embodiments, the fragment corresponds to an internal fragment that lacks both the amino and carboxyl terminal amino acids. In some embodiments, the fragment is 7-200 amino acid residues in length. In some embodiments, the fragment is 10-100 amino acid residues, 15-85 amino acid residues, 25-65 amino acid residues or 30-50 amino acid residues in length. In some embodiments, the fragment is 7 amino acids, 10 amino acids, 12 amino acids, 15 amino acids, 20 amino acids, 25 amino acids, 30 amino acids, 35 amino acids, 40 amino acids, 45 amino acids, 50 amino acids 55 amino acids, 60 amino acids, 80 amino acids or 100 amino acids in length.

In some embodiments, the fragment is at least 50 amino acids, 100 amino acids, 150 amino acids, 200 amino acids or at least 250 amino acids in length. Of course, larger antigenic fragments are also useful according to the present invention, as are fragments corresponding to most, if not all, of the amino acid sequence of the polypeptides described herein.

In some embodiments, the polypeptides have an amino acid sequence at least 80, 85%, 90%, 91%, 92%, 93%, 94%, 95%, 96%, 97%, 98%, 99%, or 100% identical to the polypeptides described herein or antigenic fragments thereof. In some embodiments, the variants are those that vary from the reference by conservative amino acid substitutions, i.e., those that substitute a residue with another of like characteristics. Typical substitutions are among Ala, Val, Leu and Ile; among Ser and Thr; among the acidic residues Asp and Glu; among Asn and Gln; and among the basic residues Lys and Arg, or aromatic residues Phe and Tyr. In some embodiments, the polypeptides are variants in which several, 5 to 10, 1 to 5, or 1 to 2 amino acids are substituted, deleted, or added in any combination.

In some embodiments, the polypeptides are encoded by polynucleotides that are optimized for high level expression in E. coli using codons that are preferred in E. coli. As used herein, a codon that is “optimized for high level expression in Salmonella” refers to a codon that is relatively more abundant in E. coli in comparison with all other codons corresponding to the same amino acid. In some embodiments, at least 10% of the codons are optimized for high level expression in E. coli. In some embodiments, at least 25%, at least 50%, at least 75%, at least 80%, at least 85%, at least 90%, at least 95%, or at least 99% of the codons are optimized for high level expression in E. coli.

In some embodiments, the polypeptide or antigenic fragment thereof comprises a cleavable protein sequence and/or affinity tag to aid in purification. In some embodiments, the affinity tag comprises at least 6 histidine residues. In some embodiments, the polypeptide or antigenic fragment thereof comprises a secretion signal to facilitate secretion of the protein through plasma membrane. In some embodiments, the secretion signal is a lysozyme secretion signal.

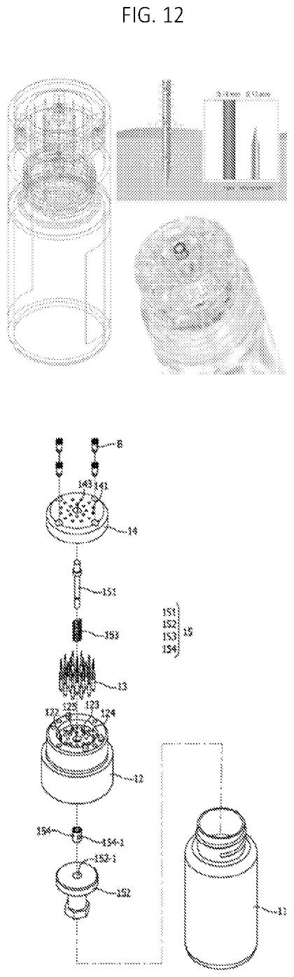

As provided herein, the composition comprising the allergen is administered using a microneedle delivery device. In some embodiments, the microneedle delivery device useful in the methods of the invention is depicted in FIG. 12. In some embodiments, the microneedle drug delivery device is as described in Korean Patent No. 10-1582822, which is incorporated by reference herein in its entirety.

In some embodiments, the microneedle delivery device comprises
i) one or more microneedles, wherein the microneedles are hollow or non-hollow, wherein one or multiple grooves are inset along an outer wall of the microneedles; and
ii) a reservoir that holds the composition to be delivered, wherein the reservoir is attached to or contains a means to encourage flow of the bioactive composition contained in the reservoir into the skin.

In some embodiments, the means to encourage flow of the composition contained in the reservoir into the skin is selected from the group consisting of a plunger, pump and suction mechanism. In some embodiments, the means to encourage flow of the composition contained in the reservoir into the skin is a mechanical spring loaded pump system.

In some embodiments, the microneedles have a single groove inset along the outer wall of the microneedle, wherein the single groove has a screw thread shape going clockwise or counterclockwise around the microneedle.

In some embodiments, the microneedles are from 0.1 mm to about 2.5 mm in length and from 0.01 mm to about 0.05 mm in diameter.

In some embodiments, the microneedles are made from a substance comprising gold.

In some embodiments, the plurality of microneedles comprises an array of microneedles in the shape of a circle.

In some embodiments, the microneedles are made of 24-carat gold plated stainless steel and comprise an array of about 10 to about 50 microneedles. In some embodiments, the array comprises 20 microneedles.

In some embodiments, the microneedle delivery device is repeatedly pressed against the subject's skin to deliver the composition to the area of the skin to be treated. In some embodiments, the microneedle delivery device is repeatedly pressed about 10, about 20, about 30, about 40, about 50, about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 or more times to administer the composition.

In some embodiments, the composition is administered by the microneedle delivery device with a repeated motion of penetrating the microneedle delivery device into the skin of the subject. In some embodiments, the composition is delivered into the skin by passing through the one or multiple grooves along the outer wall of the microneedle. In some embodiments, the microneedles are non-hollow.

In some embodiments, the administering comprises a repeated motion of penetrating the microneedle delivery device into the subject's skin in different areas of the subject's body.

In some embodiments, the subject's skin in the head, limbs and/or torso regions are repeatedly penetrated by the microneedle delivery device.

For example, repeated penetrations can be made in the subject's arms, legs, and torso in order to deliver the composition to the subject's body.

In some embodiments, the microneedle delivery device comprises a single or an array of microneedles. In some embodiments, the microneedles will have one or multiple grooves inset along its outer wall. This structural feature of the dermal delivery device allows liquids stored in a reservoir at the base of each needle to travel along the needle shaft into the tissue.

In some embodiments, the microneedle array comprises from about 1 to about 500 microneedles, which will be anywhere from about 0.1 to about 2.5 mm in length and from 0.01 to about 0.5 mm in diameter, and be composed of any metal, metal alloy, metalloid, polymer, or combination thereof, such as gold, steel, silicon, PVP (polyvinylpyrrlidone), etc. The microneedles will each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array will be in any shape or combination of shapes, continuous, or discontinuous. The list of possible shapes includes, but is not limited to, circles, triangles, rectangles, squares, rhomboids, trapezoids, and any other regular or irregular polygons. The array can be attached to a reservoir to hold the substances to be delivered, and this reservoir will be any volume (0.25 mL to 5 mL), shape, color, or material (glass, metal, alloy, or polymer), as determined necessary. This reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The microneedle delivery device is capable of delivering compositions directly to the epidermal, dermal and subcuticular layers of the skin. Therefore, it should be understood that further embodiments developed for use with non-hollow or hollow microneedle systems of delivery by those skilled in the art fall within the spirit and scope of this disclosure.

In another aspect, a microneedle device for use in the methods described herein is a device such as described in U.S. Pat. No. 8,257,324, which is hereby incorporated by reference. Briefly, the devices include a substrate to which a plurality of hollow microneedles are attached or integrated, and at least one reservoir, containing a bioactive formulation, selectably in communication with the microneedles, wherein the volume or amount of composition to be delivered can be selectively altered. The reservoir can be, for example, formed of a deformable, preferably elastic, material. The device typically includes a means, such as a plunger, for compressing the reservoir to drive the bioactive formulation from the reservoir through the microneedles, A reservoir, can be, for example, a syringe or pump connected to the substrate. A device, in some instances, comprises: a plurality of hollow microneedles (each having a base end and a tip), with at least one hollow pathway disposed at or between the base end and the tip, wherein the microneedles comprise a metal; a substrate to which the base ends of the microneedles are attached or integrated; at least one reservoir in which the material is disposed and which is in connection with the base end of at least one of the microneedles, either integrally or separably; a sealing mechanism interposed between the at least one reservoir and the substrate, wherein the sealing mechanism comprises a fracturable barrier; and a device that expels the material in the reservoir into the base end of at least one of the microneedles and into the skin. The reservoir comprises a syringe secured to the substrate, and the device that expels the material comprises a plunger connected to a top surface of the reservoir. The substrate may be adapted to removably connect to a standard or Luer-lock syringe. In one instance, the device may further include a spring engaged with the plunger. In another instance, the device may further include an attachment mechanism that secures the syringe to the device. In another instance, the device may further include a sealing mechanism that is secured to the tips of the microneedles. In another instance, the device may further include means for providing feedback to indicate that delivery of the material from the reservoir has been initiated or completed. An osmotic pump may be included to expel the material from the reservoir. One or more microneedles may be disposed at an angle other than perpendicular to the substrate. In certain instances, the at least one reservoir comprises multiple reservoirs that can be connected to or are in communication with each other. The multiple reservoirs may comprise a first reservoir and a second reservoir, wherein the first reservoir contains a solid formulation and the second reservoir contains a liquid carrier for the solid formulation. A fracturable barrier for use in the devices can be, for example, a thin foil, a polymer, a laminate film, or a biodegradable polymer. The device may further comprise, in some instances, means for providing feedback to indicate that the microneedles have penetrated the skin.

In some embodiments, the device can include, in some instances, a single or plurality of solid, screw-type microneedles, of single or varied length. Typically the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrlidone) etc. The screw-shape dimensions may be variable. For example, in one embodiment the screw-shape may be a tight coiled screw shape, whereas in another embodiment the screw-shape might be a loose coiled screw shape whereby the screw threads in one embodiment lie closely together along the outer edge of the needle and, in another embodiment, the screw threads lie far from each other along the outer edge of the needle.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

A device described herein may contain, in certain instances, about twenty screw thread design surgical grade microneedles. Each microneedle has a diameter that is thinner than a human hair and may be used for direct dermal application. In one instance, a microneedle has a diameter of less than about 0.18 mm. In another instance, a microneedle has a diameter of about 0.15 mm, about 0.14 mm, about 0.13 mm, about 0.12 mm, about 0.11 mm, or about 0.10 mm. Each microneedle may be plated with 24 carat gold. The device allows for targeted and uniform delivery of a composition comprising the immunizing composition into the skin in a process that is painless compared to injectables. Administration can result in easy and precise delivery of a composition comprising the immunizing composition with generally no bruising, pain, swelling and bleeding caused by the injection.

The device may include means, manual or mechanical, for compressing the reservoir, creating a vacuum, or otherwise using gravity or pressure to drive the immunizing composition from the reservoir through the microneedles or down along the sides of the microneedle. The means can include a plunger, pump or suction mechanism. In another embodiment, the reservoir further includes a means for controlling rate and precise quantity of drug delivered by utilizing a semi-permeable membrane, to regulate the rate or extent of drug which flows along the shaft of the microneedles. The microneedle device enhances transportation of drugs across or into the tissue at a useful rate. For example, the microneedle device must be capable of delivering drug at a rate sufficient to be therapeutically useful. The rate of delivery of the drug composition can be controlled by altering one or more of several design variables. For example, the amount of material flowing through the needles can be controlled by manipulating the effective hydrodynamic conductivity (the volumetric through-capacity) of a single device array, for example, by using more or fewer microneedles, by increasing or decreasing the number or diameter of the bores in the microneedles, or by filling at least some of the microneedle bores with a diffusion-limiting material. It can be preferred, however, to simplify the manufacturing process by limiting the needle design to two or three “sizes” of microneedle arrays to accommodate, for example small, medium, and large volumetric flows, for which the delivery rate is controlled by other means.

Other means for controlling the rate of delivery include varying the driving force applied to the drug composition in the reservoir. For example, in passive diffusion systems, the concentration of drug in the reservoir can be increased to increase the rate of mass transfer. In active systems, for example, the pressure applied to the reservoir can be varied, such as by varying the spring constant or number of springs or elastic bands. In either active or passive systems, the barrier material can be selected to provide a particular rate of diffusion for the drug molecules being delivered through the barrier at the needle inlet.

The array may be in any shape or combination of shapes, continuous, or discontinuous. The list of possible shapes includes, but is not limited to, circles, triangles, rectangles, squares, rhomboids, trapezoids, and any other regular or irregular polygons.

The array may be attached to a reservoir to hold the substances to be delivered, and this reservoir may be any volume (about 0.25 mL to about 5 mL), shape, color, or material (glass, metal, alloy, or polymer), as determined necessary.

This reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

In some embodiments, the device can include a single or plurality of solid, screw-type microneedles, of single or varied lengths housed in a plastic or polymer composite head which embodies a corrugated rubber connector. In some embodiments, the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrlidone) etc. The screw-shape dimensions may be variable. For example, in one embodiment the screw-shape may be a tight coiled screw shape, whereas in another embodiment the screw-shape might be a loose coiled screw shape. The corrugated rubber connector is a unique advantage conferring feature which bestows the microneedle head with a universally adoptable feature for interfacing the micro needle cartridges with multiple glass and or plastic vials, reservoirs and containers as well as electronic appendages for an altogether enhanced adjunct liquid handling, security and surveillance utility.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. A method for determining whether a subject is allergic to an allergen, comprising
iii) administering to the subject's skin at an injection site a composition comprising the allergen, wherein the composition is administered with a microneedle delivery device; and
iv) monitoring the injection site of the subject's skin for the appearance of a hypersensitivity reaction,
wherein the appearance of the hypersensitivity reaction indicates that the subject is allergic to the allergen.

2. The method of claim 1, wherein the allergen is from a substance shown in Table 1.

3. The method of any of claims 1-2, wherein the allergen is a polypeptide.

4. The method of any of claim 1-3, wherein the injection site is monitored for at least 15 minutes.

5. The method of any of claim 1-4, wherein the injection site is monitored for at least 30 minutes.

6. The method of any of claim 1-5, wherein the injection site is monitored for at least one hour.

7. The method of any of claims 1-6, wherein the administering comprises a repeated motion of penetrating the microneedle delivery device into the subject's skin at the injection site.

8. The method of any of claims 1-7, wherein a plurality of compositions are administered to the subject's skin at a plurality of injection sites.

9. The method of claim 8, wherein a plurality of microneedle delivery devise are used to administer the compositions.

10. The method of any of claim 8 or 9, wherein the plurality of injection sites are arranged in a grid format on the subject's skin.

11. The method of claim 10, wherein the injection sites are located on the subject's back and/or forearm.

12. The method of any one of claims 1-11, wherein the microneedle delivery device comprises
iii) one or more microneedles, wherein the microneedles are hollow or non-hollow, wherein one or multiple grooves are inset along an outer wall of the microneedles; and
iv) a reservoir that holds the composition to be delivered, wherein the reservoir is attached to or contains a means to encourage flow of the composition contained in the reservoir into the skin;
wherein the composition is delivered into the skin by passing through the one or multiple grooves along the outer wall of the microneedle.

13. The method of claim 12, wherein the microneedles are non-hollow.

14. The method of any of claim 12 or 13, wherein the means to encourage flow of the composition contained in the reservoir into the skin is selected from the group consisting of a plunger, pump and suction mechanism.

15. The method of any of claims 12-14, wherein the means to encourage flow of the composition contained in the reservoir into the skin is a mechanical spring loaded pump system.

16. The method of any of claims 12-15. wherein the microneedles have a single groove inset along the outer wall of the microneedle, wherein the single groove has a screw thread shape going clockwise or counterclockwise around the microneedle.

17. The method of any of claims 12-16, wherein the microneedles are from 0.1 mm to about 2.5 mm in length and from 0.01 mm to about 0.05 mm in diameter.

18. The method of any of claims 12-17, wherein the microneedles are made from a substance comprising gold.

19. The method of any of claims 12-18, wherein the plurality of microneedles comprises an array of microneedles in the shape of a circle.

20. The method of any of claims 12-19, wherein the microneedles are made of 24-carat gold plated stainless steel and comprise an array of 20 microneedles.

21. The method of any of claims 1-20, further comprising administering to the subject a treatment for the allergy.

22. The method of claim 21, wherein the treatment comprises administering to the subject an effective amount of an immunosuppressant.
